(19) United States
(12) Patent Application Publication
(10) Pub. No.: US 2010/0280570 A1
(43) Pub. Date: Nov. 4, 2010

(54) APPARATUS FOR THE STIMULATION OF THE SPINAL CHORD

(76) Inventors: Volker Sturm, Meckesheim (DE); Athanasios Koulousakis, Koeln (DE); Peter Tass, Muenchen (DE)

Correspondence Address: KF ROSS PC, 5683 RIVERDALE AVENUE, SUITE 203 BOX 900, BRONX, NY, 10471-0900, US

(21) Appl. No.: 12/740,179

(22) PCT Filed: Oct. 28, 2008
(86) PCT No.: PCT/DE2008/001747

§ 371 Date: Jul. 9, 2010

(30) Foreign Application Priority Data
Oct. 30, 2007 (DE) 10 2007 051 847.3

(51) Int. Cl. A61N 1/00

(52) U.S. Cl. 607/43

(57) ABSTRACT

An apparatus (100) is described that comprises a generator unit (1) of generating electrical stimulation signals and a spinal cord stimulation unit (2) encompassing a plurality of stimulation contact surfaces (11-14) to which the generator unit (1) feeds the stimulation signals. At least two of the stimulation contact surfaces (11-14) are arranged in a staggered manner transverse to the direction in which the spinal cord extends.

The invention relates to an apparatus and a method for stimulating the spinal cord by means of electric stimulation signals.

In particular, the invention relates to a stimulation apparatus and a stimulation method for the treatment of pain disorders, angina pectoris and peripheral artery occlusive disease.

Against this backdrop, an apparatus as per claim 1, a use of the apparatus claimed in claim 1 as per claim 19, an apparatus as per claim 20, a spinal cord stimulation unit as per claim 21 and a method as per claim 24 are specified. Advantageous developments and refinements of the invention are specified in the dependent claims.

In the following text, the invention will be explained in more detail in an exemplary fashion with reference to the drawings, in which:

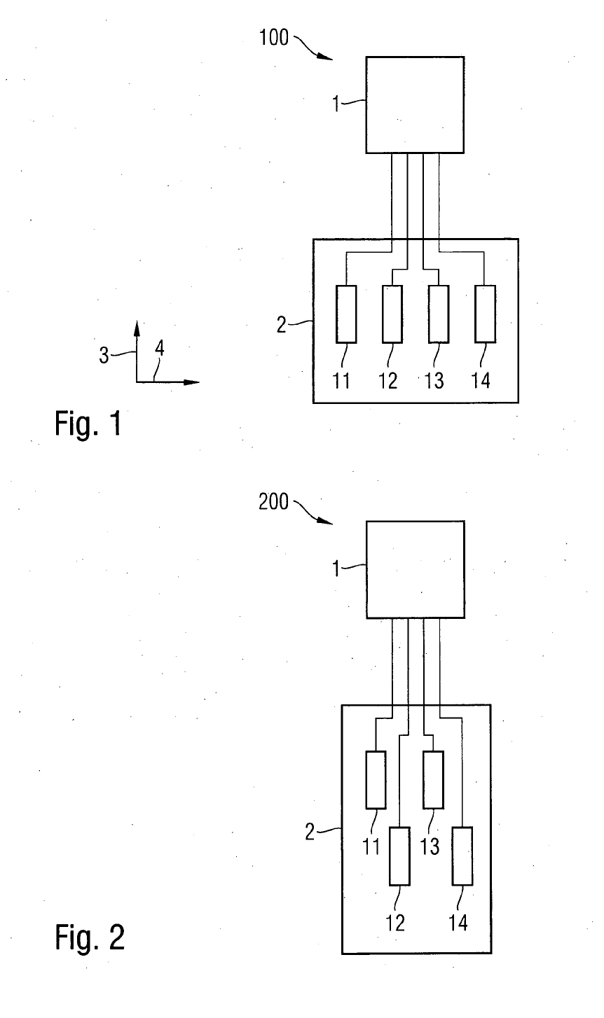

FIG. 1 shows a schematic illustration of an apparatus 100 as per an exemplary embodiment;

FIG. 2 shows a schematic illustration of an apparatus 200 as per a further exemplary embodiment;

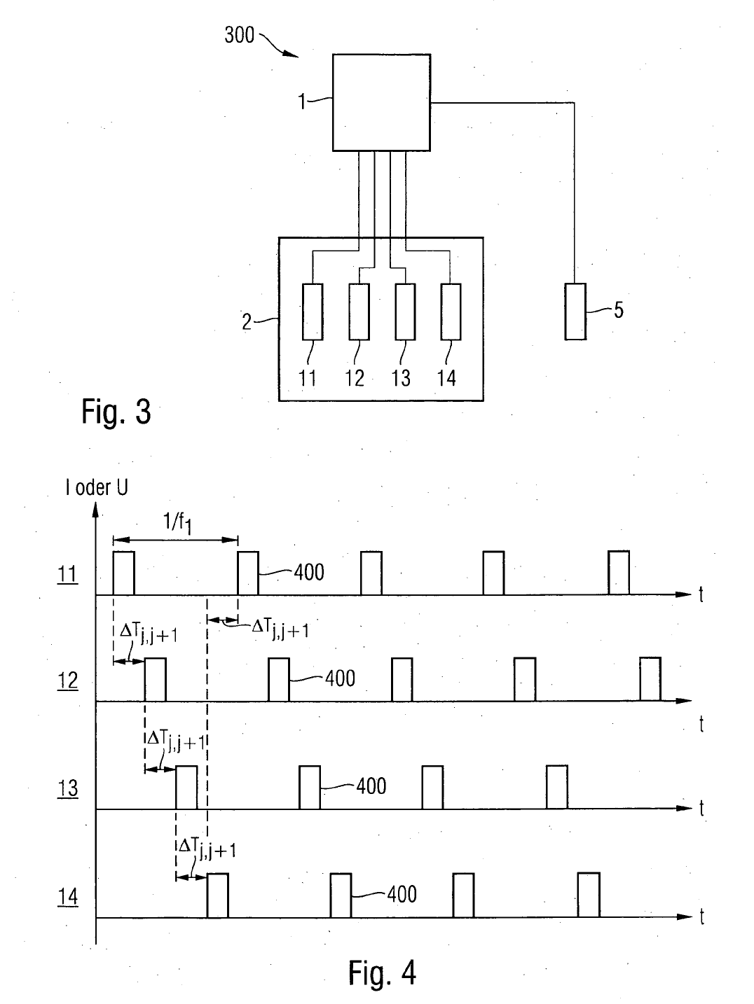

FIG. 3 shows a schematic illustration of an apparatus 300 as per a further exemplary embodiment;

FIG. 4 shows a schematic illustration of stimulation signals applied by means of a plurality of stimulation contact surfaces;

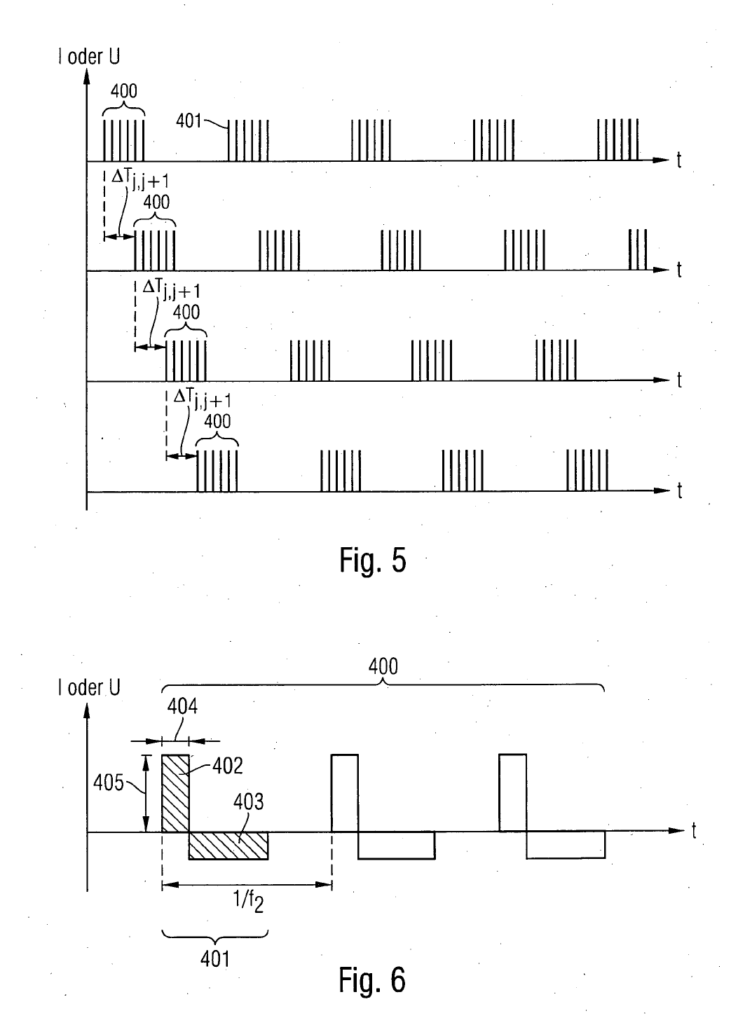

FIG. 5 shows a schematic illustration of sequences of pulse trains applied by means of a plurality of stimulation contact surfaces;

FIG. 6 shows a schematic illustration of a pulse train;

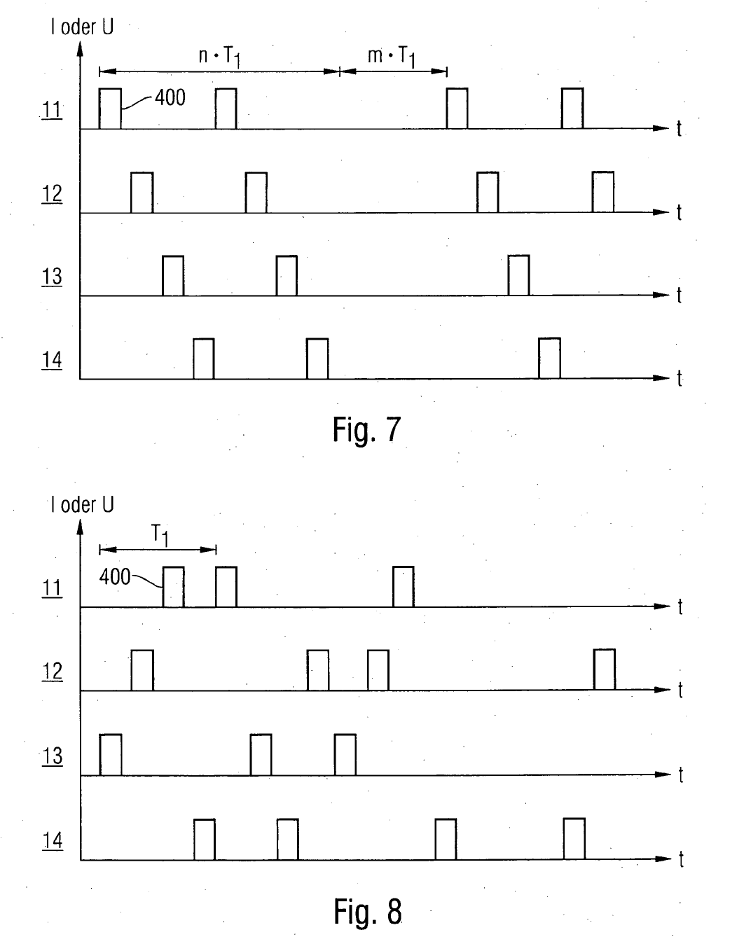

FIG. 7 shows a schematic illustration of a variation of the stimulation shown in FIG. 4;

FIG. 8 shows a schematic illustration of a further variation of the stimulation shown in FIG. 4;

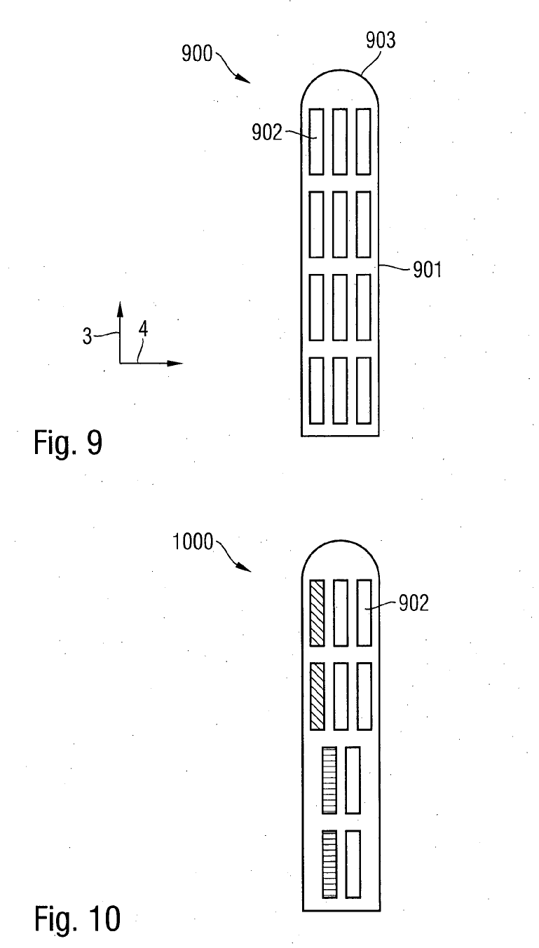

FIG. 9 shows a schematic illustration of a spinal cord stimulation unit 900 as per a further exemplary embodiment;

FIG. 10 shows a schematic illustration of a spinal cord stimulation unit 1000 as per a further exemplary embodiment;

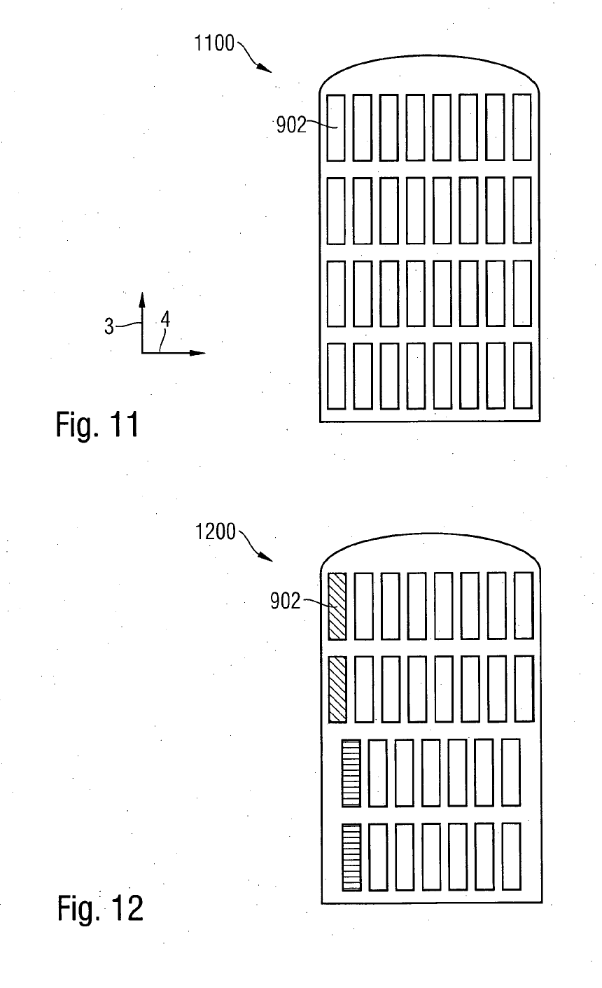

FIG. 11 shows a schematic illustration of a spinal cord stimulation unit 1100 as per a further exemplary embodiment;

FIG. 12 shows a schematic illustration of a spinal cord stimulation unit 1200 as per a further exemplary embodiment;

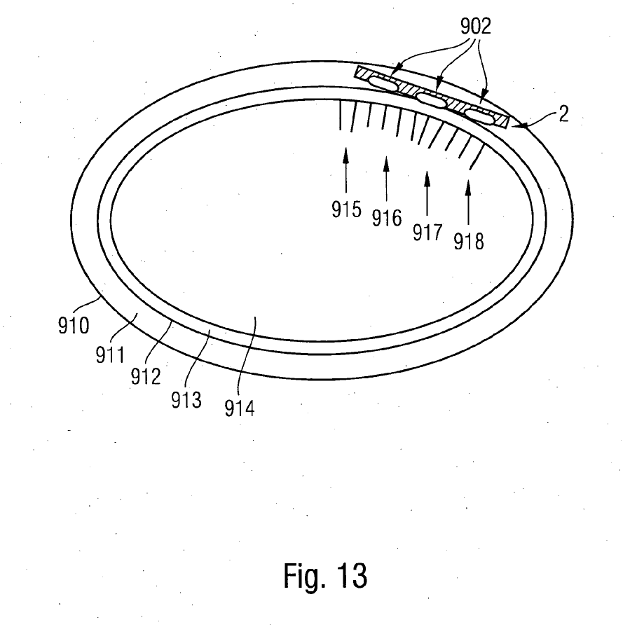

FIG. 13 shows a schematic illustration of an implanted spinal cord stimulation unit 2; and

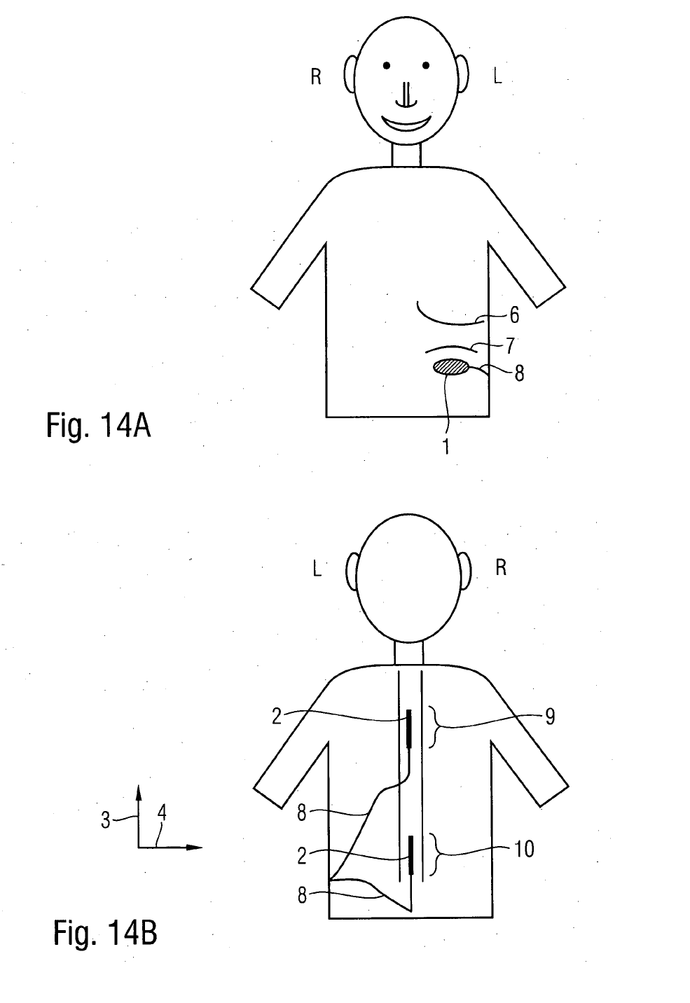

FIGS. 14A and 14B show schematic illustrations of an implanted apparatus as per a further exemplary embodiment.

FIG. 1 schematically illustrates an apparatus 100. The apparatus 100 consists of a generator unit 1 and a spinal cord stimulation unit 2 connected to the generator unit 1. The spinal cord stimulation unit 2 contains at least two stimulation contact surfaces. In the present exemplary embodiment, the spinal cord stimulation unit 2 has four stimulation contact surfaces 11, 12, 13, and 14. Furthermore, FIG. 1 specifies a first direction 3 and a second direction 4 substantially perpendicular thereto. When the spinal cord stimulation unit 2 is implanted, the latter is inserted into the body of the patient such that the direction 3 substantially corresponds to the extent of the spinal cord. The stimulation contact surfaces 11 to 14 are arranged offset along the direction 4, which substantially constitutes the transverse direction to the extent of the spinal cord.

During the operation of the apparatus 100, the generator unit 1 generates stimulation signals, which are fed into the spinal cord stimulation unit 2 and which are emitted by the stimulation contact surfaces 11 to 14 to the segments of the spinal cord that are in contact with the respective stimulation contact surfaces 11 to 14. Thus, the stimulation contact surfaces 11 to 14 each stimulate different neurons.

In the apparatus 100, the stimulation contact surfaces 11 to 14 are arranged in a row and at a distance from one another. An alternative exemplary embodiment thereto is illustrated in FIG. 2. In the apparatus 200 shown therein, the stimulation contact surfaces 11 to 14 are arranged in two rows and the stimulation contact surfaces 12 and 14 of the bottom row are arranged offset along the direction 4 with respect to the stimulation contact surfaces 11 and 13 of the upper row.

The arrangement of the stimulation contact surfaces 11 to 14 allows separate stimulation of the fibers of different segments of the spinal cord by means of the individual stimulation contact surfaces 11 to 14. The stimulation contact surfaces 11 to 14 can be arranged such that the stimulation signals applied to the fibers of the spinal cord are relayed by means of the spinal cord to different target areas, which for example are situated in the spinal cord itself or in the brain. It follows that the apparatuses 100 and 200 can be used to stimulate different target areas in the spinal cord and/or in the brain during the same stimulation period, using possibly different and/or time-shifted stimuli.

The apparatuses 100 and 200 can be used in particular for the treatment of severe, therapy-resistant pain disorders, angina pectoris and peripheral artery occlusive disease, but also for the treatment of other diseases.

In the case of severe pain disorders, can be caused by a dysfunction in the bioelectric communication of neural networks, which are combined in specific circuits. Herein, a neuron population generates continuous abnormal neural activity and abnormal connectivity (network structure) connected thereto. In the process, a large number of neurons form synchronous action potentials, i.e. the involved neurons fire in an overly synchronous fashion. Additionally, the pathological neuron population has oscillatory neural activity, i.e. there is rhythmic firing of the neurons. In the case of severe pain disorders, the average frequency of the abnormal rhythmic activity of the affected neural networks is approximately in the range between 1 and 20 Hz, but it can also lie outside of this range. By contrast, in healthy humans the neurons fire in a qualitatively different fashion, for example in an uncontrolled fashion.

In one hypothesis for explaining severe pain disorders, the assumption is made that there are pathological synchronization phenomena of neurons in the gelatinous substance. In the process, peripheral pain stimuli are relayed via the C and A 2-fiber system, firstly in peripheral nerves to the spinal ganglion and from there into the spinal cord via the dorsal roots. The switching of the pain impulses to the anterolateral columns (tractus spinothalamicus) is carried out in the gelatinous substance at the base of the posterior horns. Here there obviously are pathological synchronization phenomena of neurons, i.e. an amplification of the pain. An electric stimulus of the posterior bundle of the spinal cord, by means of which the proprioception, i.e. not the actual pain impulses themselves, is transmitted, can influence the switching zone, i.e. the gelatinous substance, and this can achieve blocking of the pain transmission.

A hypothesis for explaining the effectiveness of the spinal cord stimulation is the so-called “gait control theory”. It is based on the fact that fibers transmitting senses of touch have significantly stronger myelination and thus transmit stimuli significantly faster than the almost marrow-free so-called protopathic C fiber system, which is decisive for conducting pain. Electric stimulation of the posterior bundle mainly relates to the quickly conducting B fiber system and blocks, on the level of the gelatinous substance, the (protopathic) pain impulses arriving with relatively large temporal latency. Additionally, the stimulation of the posterior bundle activates ascending fibers and pathways, which likewise bring about a blockade of the pain transmission on a thalamic level. Descending fibers can also become active in a pain reducing fashion by electric activation in the posterior bundle region. In this case, it seems that mainly certain peptides, in particular endorphins and enkephalins and substance P, are effective.

The offset arrangement of the stimulation contact surfaces 11 to 14 in a transverse direction to the spinal cord allows the inclusion of fibers originating from adjacent segments when stimulating the spinal cord. This significantly increases the “accuracy” of the method, i.e. the influencing of the spatial pain distribution of spinal cord sections corresponding in a somatotopic fashion. Furthermore, the different segments of the spinal cord can be stimulated separately.

By way of example, the apparatuses 100 and 200 can be operated in a so-called “open loop” mode, in which the generator unit 1 generates prescribed stimulation signals and the latter are emitted to the spinal cord by the stimulation contact surfaces 11 to 14. Moreover, the apparatuses 100 and 200 can also be developed to form an apparatus 300 shown in FIG. 3, which constitutes a so-called “closed loop” system. The apparatus 300 additionally contains a measurement unit 5, which records measurement signals from nerve cells and transmits said signals to the generator unit 1. Provision can be made for the generator unit 1 to generate the stimulation signals on the basis of the measurement signals recorded by the measurement unit 4. The measurement unit 4 can be implanted into the body of the patient in the form of one or more sensors. The sensors can for example be designed as electrodes for measuring neural and/or vegetative activities, in particular as intracerebral electrodes, epicortical electrodes or subcutaneous electrodes. In particular, the measurement unit 5 can measure the physiological activity in the stimulated target region or in a region connected thereto.

Various refinements are feasible in respect of the interaction of the generator unit 1 with the measurement unit 5. By way of example, the generator unit 1 can bring about stimulation controlled by requirements. For this, the generator unit 1 detects the presence and/or the development of one or more abnormal features on the basis of the measurement signal recorded by the measurement unit 5. By way of example, the amplitude or the magnitude of the neural activity can be measured and compared to a predetermined threshold value. The generator unit 1 can be designed such that a stimulation of one or more of the aforementioned target regions is initiated as soon as the predetermined threshold value is exceeded. As an alternative to controlling the times of the stimulation on the basis of the measurement signals recorded by the measurement unit 5 or in addition thereto, the generator unit 1 can for example set the strength of the stimulation signals on the basis of the development of the abnormal features. By way of example, one or more threshold values can be predetermined, and the generator unit 1 sets a certain strength of the stimulation signals when the amplitude or the magnitude of the measurement signal exceeds a certain threshold value.

Moreover, provision can be made for the measurement signals recorded by the measurement unit 5 to be used directly, or possibly after one or more processing steps, as stimulation signals and for said measurement signals to be fed into the spinal cord stimulation unit 2 by the generator unit 1. By way of example, the measurement signals can be amplified and, if need be after mathematical calculations (for example after mixing the measurement signals), can be processed with a time delay and linear and/or nonlinear calculation steps and combinations and can be fed into at least one stimulation contact surface of the spinal cord stimulation unit 2. Herein, the calculation mode is selected such that the abnormal neural activity is counteracted and that the stimulation signal likewise disappears with reducing abnormal neural activity, or at least that the strength of the signal is reduced significantly.

Due to the offset arrangement of the stimulation contact surfaces 11 to 14, it is not only the different segments of the spinal cord that can be stimulated, but other stimulation forms than would be possible in the case of using for example only one stimulation contact surface can also be utilized. In one refinement, stimulation signals are fed into the spinal cord by the spinal cord stimulation unit 2, which signals bring about a reset in the neuron population of the phase of the neural activity of the stimulated neurons when said signals are transmitted to a neuron population with an abnormally synchronous and oscillatory activity via the spinal cord. As a result of the reset, the phase of the stimulated neurons is set to a certain phase value, for example 0°, independently of the current phase value. Thus, the phase of the neural activity of the abnormal neuron population is controlled by means of a targeted stimulation. Furthermore, as a result of the plurality of stimulation contact surfaces, it is possible to stimulate the abnormal neuron population at different places. This affords the possibility of, at the different stimulation places, resetting the phase of the neural activity of the abnormal neuron population at different times. As a result of this, the abnormal neuron population, the neurons of which were previously active in a synchronous fashion at the same frequency and phase, is split into a plurality of subpopulations. Within a subpopulation, the neurons are still synchronous and also still fire with the same pathological frequency, but, in respect of its neural activity, each of the subpopulations has the phase forced onto it by the stimulation stimulus.

Due to the abnormal interaction between the neurons, the state with at least two subpopulations generated by the stimulation is unstable, and the entire neuron population quickly approaches a state of complete desynchronization, in which the neurons fire in an uncorrelated fashion. Hence, the desired state, i.e. the complete desynchronization, is not present immediately after the application of the stimulation signals by means of the spinal cord stimulation unit 2, but is usually set within a few periods or even within less than one period of the pathological frequency.

In the type of stimulation described above, the ultimately desired desynchronization is only made possible by the abnormally increased interaction between the neurons. Hereby, a self-organization process is utilized, which is responsible for the abnormal synchronization. The same process brings about a desynchronization following a subdivision of an entire population into subpopulations with different phases. In contrast to this, there would be no desynchronization without an abnormally increased interaction of the neurons.

Moreover, the stimulation using the apparatuses 100 to 300 obtained a reorganization of the connectivity of the dysfunctional neural networks, and so long-term therapeutic effects are brought about.

If different stimulation signals were used instead of the stimulation signals by means of which the phases of the stimulated neurons could be controlled, for example high-frequency, continuously applied high-frequency pulse trains, the long-term therapeutic effects described above may no longer be obtained and this would lead to continuous and relatively high-current stimulations being necessary. In contrast to this, the stimulation forms described herein only require little energy to be introduced into the neural system from the outside in order to achieve a therapeutic effect.

Electro-stimulation of the spinal cord can cause uncomfortable dysesthesia or paresthesia in the patient or else can be accompanied by severe pains, which occur particularly when the epidurally (i.e. between the dura mater surrounding the spinal cord and the bony vertebral canal) placed electrodes come into contact with nerve roots. The strength of the dysesthesiae or paresthesia increases as the voltage or current strength used increases. Due to the above-described relatively low energy input into the spinal cord and the frequently very quickly obtained stimulation results, the dysesthesiae and paresthesiae accompanying the stimulation can be significantly reduced by the apparatuses 100 to 300.

Furthermore, the stimulation described herein requires relatively little current, as a result of which an operative change of the generator unit 1 only has to be carried out infrequently.

Different procedures can be implemented in order to obtain a desynchronization of the entire neuron population due to time-shifted resetting of the phase of subpopulations of an abnormally synchronous neuron population. By way of example, stimulation signals that bring about a resetting of the phase of neurons can be emitted in a time-shifted fashion by the different stimulation contact surfaces 11 to 14 to the respectively stimulated segments of the spinal cord. Moreover, the stimulation signals can, for example, be applied with a phase shift or with different polarity, and so ultimately they also lead to a time-shifted resetting of the phases of the different subpopulations.

A stimulation method suitable for the above-described purposes, which can for example be brought about by one of the apparatuses 100 to 300, is illustrated schematically in FIG. 4. In FIG. 4, the stimulation signals 400 applied by the stimulation contact surfaces 11 to 14 are plotted, one below the other, against the time t.

In the method illustrated in FIG. 4, each of the stimulation contact surfaces 11 to 14 applies the stimulation signal 400 to the respective segment of the spinal cord in a periodic fashion. The frequency f₁ at which the stimulation signals 400 are repeated per stimulation contact surface 11 to 14 can lie in the range between 1 and 30 Hz and in particular in the range between 5 and 20 Hz, although it can also assume smaller or larger values.

As per the refinement shown in FIG. 4, the stimulation signals 400 are dispensed by the individual stimulation contact surfaces 11 to 14 with a time delay between the individual stimulation contact surfaces 11 to 14. By way of example, the start of temporally successive stimulation signals, applied by different stimulation contact surfaces 11 to 14, can be displaced by a time ΔT_{j,j+1}.

In the case of N stimulation contact surfaces, the time delay ΔT_{j,j+1} between two respectively successive stimulation signals 400 can for example lie in the region of an N-the of the period 1/f₁. In the exemplary embodiment shown in FIG. 4, the time delay ΔT_{j,j+1} then is 1/(4×f₁).

The frequency f₁ can for example lie in the region of the average frequency of the abnormally rhythmic activity of the target network. In the case of severe therapy-resistant pain disorders, the average frequency typically lies in the range between 1 and 20 Hz, but it can also lie outside of this range. Here, it should be taken into account that the frequency at which the affected neurons fire synchronously in the aforementioned diseases generally is not constant but can by all means have variations and, moreover, exhibits individual deviations in each patient.

The stimulation method described above, in which the phase is reset with a time shift at different places of a neuron population, can also be used for the treatment of angina pectoris or peripheral artery occlusive disease. As per a hypothesis in respect of the therapeutic mechanism of the stimulation method in these diseases, the stimulation disrupts the vicious cycle consisting of pain, which causes a sympathetic-nerve activation, and thus causes a stenosis in the vessel, which in turn causes pain. Furthermore, it is assumed that the stimulation brings about a desynchronizing effect on sympathetic cells of the lateral funiculus of the spinal cord (Th1-L4), by means of which the vessel stenosis is directly counteracted.

By way of example, current- or voltage-controlled pulses can be used as stimulation signals 400. Furthermore, a stimulation signal 400 can be a pulse train as illustrated in FIG. 5 consisting of a plurality of individual pulses 401. The pulse trains 400 can in each case consist of 1 to 100, in particular 2 to 10, electric charge-balanced individual pulses 401. The pulse trains 400 are applied as for example a sequence of up to 20 or more pulse trains. Within one sequence, the pulse trains 400 are repeated at the frequency f₁ within the range of 1 to 30 Hz.

A pulse train 400 consisting of three individual pulses 401 is shown in an exemplary fashion in FIG. 6. The individual pulses 401 are repeated at a frequency f₂ in the range between 50 and 500 Hz, in particular in the range between 100 and 150 Hz. The individual pulses 401 can be current- or voltage-controlled pulses that are composed of an initial pulse component 402 and a subsequent pulse component 403 flowing in the opposite direction, wherein the polarity of the two pulse components 402 and 403 can also be interchanged compared to the polarity shown in FIG. 6. The duration 404 of the pulse component 402 lies in the range between 1 μs and 450 μs. In the case of current-controlled pulses, the amplitude 405 of the pulse component 402 lies in the range between 0 mA and 25 mA, and, in the case of voltage-controlled pulses, the amplitude is in the range between 0 and 20 V. The amplitude of the pulse component 403 is smaller than the amplitude 405 of the pulse component 402. In return, the duration of the pulse component 403 is longer than that of the pulse component 402. The pulse components 402 and 403 are ideally dimensioned such that the charge transferred thereby is the same in both pulse components 402 and 403, i.e. the areas shaded in FIG. 6 are of the same size. As a result of this, an individual pulse 401 introduces the same amount of charge into the tissue as is taken from the tissue.

The rectangular shape of the individual pulses 401 illustrated in FIG. 6 represents an ideal shape. There is a deviation from the ideal rectangular shape depending on the quality of the electronics generating the individual pulses 401.

Instead of pulse-shaped stimulation signals, the generator unit 1 can for example also generate differently shaped stimulation signals, for example temporally continuous stimulus patterns. The above-described signal shapes and the parameters thereof should only be understood as being exemplary. Provision can by all means be made for there to be deviation from the aforementioned signal shapes and the parameters thereof.

There can be different deviations from the strictly periodic stimulation pattern shown in FIG. 4. By way of example, the time delay ΔT_{j,j+1} between two successive stimulation signals 400 does not necessarily always have to be the same. Provision can by all means be made for the time intervals between the individual stimulation signals 400 to be selected differently. Furthermore, the delay times can also be varied during the treatment of a patient. The delay times can also be adjusted in respect of the physiological signal run times.

Furthermore, pauses can be provided during the application of the stimulation signals 400, during which pauses there is no stimulation. Such a pause is shown in FIG. 7 in an exemplary fashion. The pauses can be selected to have an arbitrary length and can in particular consist of an integer multiple of the period T₁ (=1/f₁). Furthermore, the pauses can be observed after an arbitrary number of stimulations. For example, a stimulation can be carried out during n subsequent periods of length T₁, and subsequently a pause with no stimulation can be observed during m periods of length T₁, wherein n and m are small integers, for example in the range between 1 and 10. This scheme can either be continued periodically, or can be modified stochastically and/or deterministically, for example chaotically.

A further possibility for deviating from the strictly periodic stimulation pattern shown in FIG. 4 consists of varying the temporal sequence of the individual stimulation signals 400 stochastically or deterministically or in a mixed stochastic-deterministic fashion.

Moreover, the sequence in which the stimulation contact surfaces 11 to 14 apply the stimulation signals 400 can be varied for each period T₁ (or else in other time steps), as is shown in an exemplary fashion in FIG. 8. This variation can be brought about stochastically or deterministically or in a mixed stochastic-deterministic fashion.

Furthermore, only a certain number of stimulation contact surfaces 11 to 14 can be used for the stimulation in each period T₁ (or in a different time interval) and the stimulation contact surfaces involved in the stimulation can be varied in each time interval. This variation can also be brought about stochastically or deterministically or in a mixed stochastic-deterministic fashion.

All the above-described stimulation forms can also be carried out in a “closed loop” mode by means of the apparatus 300. In respect of the stimulation form shown in FIG. 7, the start time and the length of the pause can for example be selected controlled by requirements.

Furthermore, it is feasible for the stimulation to be started by the patient, for example by telemetric activation. In this case, the patient can activate the stimulation for a predetermined period of for example 5 minutes, for example by means of an external transmitter, or the patient can independently start and stop the stimulation.

FIG. 9 schematically illustrates the front view of an electrode 900, as can be used, for example, as a spinal cord stimulation unit 2. The electrode 900 consists of an electrically insulated electrode shaft 901 and at least two stimulation contact surfaces 902, which have been introduced into the electrode shaft 901. The stimulation contact surfaces 902 are made of an electrically conductive material, for example a metal, and are in direct electric contact with the nerve tissue of the spinal cord after the implantation. Each of the stimulation contact surfaces 902 can be actuated via its own input lead, or the recorded measurement signals can be conducted away via the input leads. The input leads are not illustrated in FIG. 9. Provision can also be made for two or more stimulation contact surfaces 902 to be actuated by a single input lead.

In the present example, the electrode 900 has four rows of stimulation contact surfaces 902 along the first direction 3. In the implanted state, the direction 3 substantially corresponds to the extent of the spinal cord. Each of the four rows has three stimulation contact surfaces 902, which are arranged at a distance from one another along the second direction 4.

The embodiment shown in FIG. 9 should merely be understood as an example. The number of rows and the number of the stimulation contact surfaces 902 provided per row can be selected differently.

The electrode tip 903, i.e. the one end of the electrode 900 along the direction 3, is rounded off to avoid damage to the tissue. The electrode 900 can be implanted in a minimally invasive, percutaneous fashion, i.e. by means of a puncture of the epidural space. The surface of the electrode 900 shown in FIG. 9 can have a planar or else a curved design. All stimulation contact surfaces 902 should be in contact with the spinal cord tissue after the implantation.

In addition to its function as spinal cord stimulation unit 2, the electrode 900 can also be utilized as a measurement unit 5. In this case, measurement signals are recorded by at least one of the contact surfaces 902.

The stimulation contact surfaces 902 can be connected to the generator unit 1 via a cable or via telemetric connections.

The length of the electrode 900 (in the direction 3) is for example 2.5 to 4 cm, and the cross-sectional diameter of the electrode 900 (in the direction 4) is for example 1 to 4 mm. The width of the stimulation contact surfaces 902 is for example 0.3 to 1 mm; the length thereof is for example 2 to 3 mm. The distances between the stimulation contact surfaces 902 in a row in the direction 4 are for example 0.1 to 0.5 mm and the distances between the rows spaced apart from one another in the direction 3 are for example 0.1 to 1 mm.

Both the number and the arrangement of the stimulation contact surfaces 902 can be selected in a fashion deviating from the refinement shown in FIG. 9. Only at least two of the stimulation contact surfaces 902 should be arranged offset in the direction 4 perpendicular to the extent of the spinal cord such that different segments of the spinal cord can be actuated with different stimulation signals. In the case of the electrode 900, the stimulation contact surfaces 902 are arranged offset and at a distance within one row. The offset arrangement of the stimulation contact surfaces 902 in the direction 4 across the extent of the spinal cord affords separate stimulation of different fibers of the spinal cord guiding the proprioception.

Moreover, the geometry of the stimulation contact surfaces 902 can be selected such that it deviates from FIG. 9. By way of example, the stimulation contact surfaces 902 do not need to have a rectangular shape, but can have for example a round or a different geometric shape.

A variation of the electrode 900 is shown in FIG. 10. The stimulation contact surfaces 902 of the bottom two rows of the electrode 1000 illustrated schematically in FIG. 10 are arranged offset to the stimulation contact surfaces 902 of the upper two rows. By way of example, the stimulation contact surfaces 902 of the bottom two rows are each arranged centrally between two stimulation contact surfaces 902 of the upper rows. Provision can be made for the electrode 1000 to have additional stimulation contact surfaces 902 and for the pattern shown in FIG. 10 to be continued periodically. As shown in FIG. 10, the bottom two rows can be equipped with a fewer number of stimulation contact surfaces 902.

Compared to the electrode 900, the electrode 1000 can be used to stimulate the individual horizontal spinal cord components associated with different body parts in a more targeted fashion, i.e. with a horizontal spatial resolution improved by half. Provision can by all means be made for the stimulation contact surfaces of the bottom two rows respectively to have an overlap with the stimulation contact surfaces 902 of the upper two rows in the direction 4.

Depending on the selection of the reference, stimulation can be carried out either in a unipolar fashion between an individual stimulation contact 902 and an electric reference potential, for example the potential of the generator unit 1, or in a bipolar fashion between two different stimulation contacts. In the case of the electrode 1000, there is bipolar stimulation via in each case two stimulation contact surfaces 902 arranged above one another in adjacent rows. FIG. 10 denotes two such pairs by corresponding shadings.

FIG. 11 shows a schematic drawing of the front view of a multi-contact electrode 1100, which is implanted under microscopic control and by means of hemilaminectomy (partial removal of the vertebral arches) or laminectomy (removal of the vertebral arches) and is directly fixed on the dura mater. The length of the electrode 1100 is for example 2.5 to 4 cm. The cross-sectional diameter of the electrode 1100 is for example 7 to 12 mm. The individual stimulation contact surfaces 902 can be designed like in the electrode 900 shown in FIG. 9. FIG. 9 shows four rows with stimulation contact surfaces 902. However, the multi-contact electrode 1100 can have an arbitrary number of rows with stimulation contact surfaces 902, for example 6 or 8 rows.

FIG. 12 shows the front view of an electrode 1200, in which two rows of stimulation contact surfaces 902 are arranged offset to other rows. By way of example, the bottom two rows can be displaced by half a horizontal contact distance with respect to the upper two rows in order to stimulate the individual horizontal spinal cord components associated with different body parts in a more targeted fashion, i.e. with a horizontal spatial resolution improved by half. Herein, it is not important whether the upper or bottom rows are offset. Furthermore, two pairs of stimulation contact surfaces are denoted in FIG. 12 by different types of shading, with bipolar stimulation being possible by these in each case.

FIG. 13 shows a schematic cross section through the cervical spinal cord and the position of a spinal cord stimulation unit 2. The spinal cord stimulation unit 2, for example one of the electrodes 900 to 1200, is in the epidural space 911, i.e. between the sack made of dura mater 912 and the connective tissue lining of the bony channel 910. Between the spinal cord stimulation unit 2 and the spinal cord 914 there is, surrounded by the dura maters, the liquor-filled subarachnoid space 913, which washes around the spinal cord 914. The stimulation contact surfaces 902 arranged perpendicularly to the longitudinal axis of the spinal cord stimulation unit 2 reach a plurality of adjacent spinal cord segments 915 to 918 at one level, which segments—as illustrated schematically—can reach a large proportion of the fibers ascending from all spinal cord segments in the case of the cervical spinal cord. The arrows 915 to 918 symbolize the fiber layers, arranged from medial to lateral, made of the sacral part of the spinal cord 915, the lumbar part of the spinal cord 916, the thoracic part of the spinal cord 917 and the sacral part of the spinal cord 918. In the case of the upper lumbar and the lower thoracic part of the spinal cord most often in question for the spinal cord stimulation, not only the fibers from the actual pain area, but also additionally fibers from the adjacent segments are reached at one level by this arrangement.

In FIGS. 14A (front view of the patient, the left and right body halves of whom are denoted respectively by “L” and “R”) and 14B (back view of the patient), the apparatus 100 (or 200) is illustrated during its intended operation. The generator unit 1 is situated in a subcutaneous recess between fascia and skin. The scar 7 for the surgical introduction of the generator unit 1 is situated approximately 3 to 4 cm below the left costal arch 6. The generator unit 3 is connected to the spinal cord stimulation unit 2 by means of one or more connection cables 8. The spinal cord stimulation unit 2 is situated in for example the lower cervical region 9 or in the lower thoracic region 10. The connection cables 8 run under the skin to the generator unit 1; if need be they are connected to the generator unit 1 by means of a connector. Furthermore, a percutaneous (leading through the skin) stimulus extension (cable conducting outward) can be provided for the test phase after the implantation of the spinal cord stimulation unit 2 and before the implantation of the generator unit 1. In the case of “closed loop” stimulation, the apparatus 900 still contains at least one sensor.

The generator unit 1 can contain control electronics which implement the stimulation methods. The generator unit 1 can comprise a long-life battery or a rechargeable accumulator as a source of energy. In an alternative refinement, the generator unit 1 can be a semi-implant with an energy source located outside of the body. Control elements in this embodiment can be located in both the implanted part and the external part of the semi-implant. The generator unit 1 can have a safety switch that ensures that safety limits, such as for example a maximum tolerable charge intake, known to a person skilled in the art are not exceeded.

1-25. (canceled)

26. An apparatus comprising:
a generator unit for generating electric stimulation signals, and
a spinal cord stimulation unit with a plurality of stimulation contact surfaces, which are fed the stimulation signals by the generator unit,
wherein
the spinal cord stimulation unit has a planar surface, on which the stimulation contact surfaces are arranged,
the stimulation contact surfaces are arranged in a plurality of rows, wherein the rows extend in a transverse direction to the extent of the spinal cord and the stimulation contact surfaces are in each row arranged at a distance from one another in a transverse direction to the extent of the spinal cord,
adjacent rows of stimulation contact surfaces are arranged at a distance from one another in the direction of the extent of the spinal cord, and
the stimulation contact surfaces of a first row of the rows of stimulation contact surfaces are in a transverse direction to the extent of the spinal cord, arranged offset to the stimulation contact surfaces of a second row of the rows of stimulation contact surfaces.

27. The apparatus defined in claim 26, wherein the generator unit feeds the stimulation contact surfaces with the stimulation signals such that the stimulation signals are emitted by the two offset stimulation contact surfaces in a time-shifted or phase-shifted fashion, or with different polarity.

28. The apparatus defined in claim 26, wherein the stimulation signals are formed such that they reset the phase of the neural activity of the stimulated neurons when stimulating neurons having an abnormally synchronous and oscillatory neural activity.

29. The apparatus defined in claim 26, wherein N of the stimulation contact surfaces emit the stimulation signals in a time-shifted or phase-shifted fashion and the offset between two respectively successive stimulation signals is on average 1/(f₁*N) is a frequency in the range between 1 and 20 Hz.

30. The apparatus defined in claim 29, wherein the frequency f₁ substantially corresponds to the average frequency of the abnormally synchronous and oscillatory neural activity of the stimulated neurons.

31. The apparatus defined in claim 29, wherein the generator unit suspends feed of stimulation signals to the spinal cord stimulation unit for a period, which lasts at least 1/f₁, after a number of stimulation signals have been fed into the spinal cord stimulation unit.

32. The apparatus defined in claim 31, wherein the period during which the feed of stimulation signals to the spinal cord stimulation unit is suspended is an integer multiple of 1/f₁.

33. The apparatus defined in claim 26, wherein the time interval between successive emission of stimulation signals via different stimulation contact surfaces is varied stochastically or deterministically or in a mixed stochastic-deterministic fashion.

34. The apparatus defined in claim 26, wherein the order of the stimulation contact surfaces to which the stimulation signals are successively fed is varied.

35. The apparatus defined in claim 26, wherein a number of the stimulation contact surfaces, which are fed stimulation signals, is selected, and the selected stimulation contact surfaces are varied.

36. The apparatus defined in claim 26, wherein the apparatus comprises a measurement unit for recording measurement signals from neurons.

37. The apparatus defined in claim 36, wherein the generator unit feeds the measurement signals into the spinal cord stimulation unit as stimulation signals or said generator unit processes the measurement signals further and feeds the further processed measurement signals into the spinal cord stimulation unit as stimulation signals.

38. The apparatus defined in claim 36, wherein the generator unit makes a decision, as a function of the measurement signals, as to whether stimulation signals are fed into the spinal cord stimulation unit.

39. The apparatus defined in claim 36, wherein the generator unit determines a parameter of the stimulation signals, in particular the strength of the stimulation signals, as a function of the measurement signals.

40. The apparatus defined in claim 26, wherein the stimulation signals are pulse trains in each case.

41. The apparatus defined in claim 26, wherein each of the stimulation contact surfaces of the second row are each arranged centrally between two stimulation contact surfaces of the first row.

42. A spinal cord stimulation unit with a plurality of stimulation contact surfaces, wherein
the spinal cord stimulation unit has a planar surface, on which the stimulation contact surfaces are arranged,
the stimulation contact surfaces are arranged in a plurality of rows, wherein the rows extend in a transverse direction to the extent of the spinal cord and the stimulation contact surfaces are in each row arranged at a distance from one another in a transverse direction to the extent of the spinal cord,
adjacent rows of stimulation contact surfaces are arranged at a distance from one another in the direction of the extent of the spinal cord, and
the stimulation contact surfaces of a first row of the rows of stimulation contact surfaces are in a transverse direction to the extent of the spinal cord, arranged offset to the stimulation contact surfaces of a second row of the rows of stimulation contact surfaces.

43. The spinal cord stimulation unit defined in claim 42, wherein the spinal cord stimulation unit is rounded off at one end.
